(19) United States
(12) Patent Application Publication
(10) Pub. No.: US 2025/0346641 A1
(43) Pub. Date: Nov. 13, 2025

(54) COMPOSITIONS AND METHODS FOR THIN FILAMENT MODULATION

(71) Applicant: ARIZONA BOARD OF REGENTS ON BEHALF OF THE UNIVERSITY OF ARIZONA, Tucson, AZ (US)

(72) Inventors: Romi Castillo, Tucson, AZ (US); Jil C. Tardiff, Tucson, AZ (US)

(21) Appl. No.: 19/202,508

(22) Filed: May 8, 2025

Related U.S. Application Data
(60) Provisional application No. 63/644,761, filed on May 9, 2024.

(51) Int. Cl. C07K 14/47; D01F 1/06; D01F 4/00

(52) U.S. Cl. CPC C07K 14/4716; D01F 1/06; D01F 4/00; D10B 2401/20; D10B 2509/00

(57) ABSTRACT

High-throughput screening (HTS) assays described herein rely on structural alterations triggered by cTnI phosphorylation during beta-adrenergic stimulation. These HTS assays may be used to pinpointsmall molecules that emulate phosphorylation effects, amplifying lusitropy by accelerating calcium dissociation from thin filaments. With a focus on addressing diastolic dysfunction, especially in conditions like HCM, the aim is to determine compounds tailored to modulate diastolic performance.

### CROSS-REFERENCE TO RELATED APPLICATIONS

This application is a non-provisional and claims benefit of U.S. Provisional Application No. 63/644,761 filed May 9, 2024, the specification(s) of which is/are incorporated herein in their entirety by reference.

### FIELD OF THE INVENTION

The present invention features compositions and methods for detecting thin filament modulation.

### BACKGROUND OF THE INVENTION

During beta-adrenergic stimulation, Protein Kinase A phosphorylates various cardiac proteins to modulate contraction and relaxation. Among these, phosphorylation of cardiac troponin I (cTnI) at site cTnI-Ser23/24 primarily enhances lusitropy. TR-FRET data on the N-terminus of cardiac troponin I indicate that phosphorylation of cTnI-Ser23/24 induces significant structural changes. This structural alteration leads to a rapid increase in calcium dissociation rate from the thin filament, driving the observed enhancement in lusitropy in vivo. Thus, the present invention features a structural-based high-throughput screening assay to identify small molecules that replicate the functional effects of phosphorylation. The goal is to discover a small molecule capable of specifically modulating diastolic performance, with the ultimate aim of treating diseases such as HCM, which are characterized by diastolic dysfunction.

### BRIEF SUMMARY OF THE INVENTION

It is an objective of the present invention to provide systems, compositions, and methods that allow for the detection of thin filament relaxation and contraction, as specified in the independent claims. Embodiments of the invention are given in the dependent claims. Embodiments of the present invention can be freely combined with each other if they are not mutually exclusive.

In some embodiments, the present invention features a recombinant protein comprising a modified human cardiac troponin protein comprising a modification at A28 and a fluorescent dye conjugate to said modification. Likewise, in some embodiments, the present invention features a recombinant protein comprising a modified human cardiac troponin protein comprising a cysteine substitution at A28 and a fluorescent dye conjugate to the cysteine substitution. In some embodiments, the recombinant protein is sensitive to phosphorylation at S2324.

In certain embodiments, the human cardiac troponin protein may further comprise a modification at cysteine 80 (C80), cysteine 97 (C97), or a combination thereof. For example, the cysteine may be substituted for a serine, an isoleucine, or the like.

Non-limiting examples of fluorescent dyes that may be used in accordance with the present invention include Tetramethylrhodamine-6-maleimide (TMR), fluorescein-5-maleimide (FMAL), N,N′-dimethyl-N-(iodoacetyl)-N′-(7-nitrobenz-2-oxa-1,3-diazol-4-yl)ethylenediamine (IANBD), or the like.

In some embodiments, the present invention features a recombinant protein comprising a modified human cardiac troponin protein comprising a modification at A28 and an N,N′-dimethyl-N-(iodoacetyl)-N′-(7-nitrobenz-2-oxa-1,3-diazol-4-yl) ethylenediamine (IANBD) probe conjugate to said modification. Likewise, in some embodiments, the present invention features a recombinant protein comprising a modified human cardiac troponin protein comprising a cysteine substitution at A28 and an N,N′-dimethyl-N-(iodoacetyl)-N′-(7-nitrobenz-2-oxa-1,3-diazol-4-yl) ethylenediamine (IANBD) probe conjugate to the cysteine substitution. In some embodiments, the recombinant protein is sensitive to phosphorylation at S2324.

In some embodiments, the present invention may feature a reconstituted complete thin filament system comprising a recombinant protein, as described herein. For example, the recombinant protein may comprise a modified human cardiac troponin protein comprising a modification at A28 (e.g., a cysteine substitution) and a fluorescent dye conjugate to said modification. In certain embodiments, the systems herein may further comprise an actin protein, a tropomyosin protein, or a combination thereof. In other embodiments, the present invention may feature a reconstituted complete thin filament system comprising a recombinant protein as described herein, an actin protein, and a tropomyosin protein. In some embodiments, the complete thin filament system responds to biological cues.

In some embodiments, the physiological interactions within the reconstituted complete thin filament system are intact.

One of the unique and inventive technical features of the present invention is the use of a modified troponin protein probe within a reconstructed thin filament system. Without wishing to limit the invention to any theory or mechanism, it is believed that the technical feature of the present invention advantageously provides for a comprehensive examination of thin filament modulation (e.g., relaxation) through molecular allostery in response to a drug or small molecule. The modified proteins and systems described herein allow for the detection of drugs binding to any portion of the thin filament. None of the presently known prior references or works have the unique inventive technical feature of the present invention.

Moreover, the prior references teach away from the present invention. For example, many systems concentrate solely on troponin in examining thin filament relaxation; however, the present invention distinguishes itself by reconstructing a comprehensive thin filament system. This system encompasses a modified troponin protein, an actin protein, and a tropomyosin protein, providing a more comprehensive perspective.

Furthermore, the inventive technical features of the present invention contributed to a surprising result. For example, assays described herein were highly successful at identifying small molecules that alter the rate of calcium dissociation from the thin filament, suggesting that structural changes to the N-terminus of cTnI frequently result in altered calcium dissociation kinetics.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic of the various cardiac thin filament systems tested with either sites cTnI-A17C or cTnI-A28C labeled with fluorescent probes IANBD, TMR, or FMAL.

FIG. 2 shows a representative fluorescence lifetime population of cTnI-A17C-FMAL WT CTF system or phosphorylated cTnI-A17C-FMAL CTF system in the presence of 0.01% DMSO. Dashed line represents the mean of the lifetime population, and solid lines represent 3 times the standard deviation of the lifetime population.

FIG. 3 shows a representative fluorescence lifetime population of cTnI-A17C-TMR WT CTF system or phosphorylated cTnI-A17C-TMR cTF system in the presence of 0.01% DMSO. Dashed line represents the mean of the lifetime population, and solid lines represent 3 times the standard deviation of the lifetime population.

FIG. 4 shows a representative fluorescence lifetime population of cTnI-A17C-IANBD WT CTF system or phosphorylated cTnI-A17C-IANBD cTF system in the presence of 0.01% DMSO. Dashed line represents the mean of the lifetime population, and solid lines represent 3 times the standard deviation of the lifetime population.

FIG. 5 shows a representative fluorescence lifetime population of cTnI-A28C-FMAL WT cTF system or phosphorylated cTnI-A28C-FMAL cTF system in the presence of 0.01% DMSO. Dashed line represents the mean of the lifetime population, and solid lines represent 3 times the standard deviation of the lifetime population

FIG. 6 shows a representative fluorescence lifetime population of cTnI-A28C-TMR WT CTF system or phosphorylated cTnI-A28C-TMR cTF system in the presence of 0.01% DMSO. Dashed line represents the mean of the lifetime population, and solid lines represent 3 times the standard deviation of the lifetime population.

FIG. 7 shows a representative fluorescence lifetime population of cTnI-A28C-IANBD WT cTF system or phosphorylated cTnI-A28C-IANBD cTF system in the presence of 0.01% DMSO. Dashed line represents the mean of the lifetime population, and solid lines represent 3 times the standard deviation of the lifetime population.

FIG. 8 shows fluorescence lifetimes from Selleck FDA-approved drug library. Solid lines represent 5 times the standard deviation of the lifetimes.

FIG. 9 shows calcium dissociation rates for the small molecule hits from primary screening using the cTnI-A28C-IANBD TR-F assay.

FIG. 10 shows a TR-F based primary screen using TnI-A28C-IANBD cardiac thin filaments with a hit cut off of ±2×SD. The TR-F based screening assay resulted in a 3% hit rate when a 2 times the standard deviation of the mean lifetime selection was applied.

FIG. 11 shows calcium dissociation rates plotted as percent change from DMSO control. All hits from the TR-F based screening assay were tested using stopped flow calcium dissociation kinetics to obtain calcium dissociation rates as a secondary screening assay. Mostsmall molecule hits altered the calcium dissociation rate by more than 10%. Small molecules that accelerated calcium dissociation rates were then subjected to tertiary screening via NADH-coupled ATPase measurements.

FIG. 12 shows NADH-coupled ATPase measurements of non-transgenic and cTnT-R 92L left ventricular myofibrils, utilized as a tertiary screen. Briefly, left ventricular myofibrils were isolated from non-transgenic and cTnT-R92L murine models. NADH-coupled ATPase measurements in the presence and absence of drug were performed. Measurements indicate that small molecule hits from the secondary screen can reduce myofibril ATPase rates in non-transgenic myofibrils and in HCM disease model myofibrils.

### DETAILED DESCRIPTION OF THE INVENTION

Disclosed are various peptides, solvents, solutions, carriers, and/or components to be used to prepare compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, while specific reference of each various individual and collective combination and permutation of these compounds may not be explicitly disclosed, each is specifically contemplated and described herein. It is also to be understood that the terminology used herein is for the purpose of describing particular embodiments only and is not intended to be limiting.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

The terms “polypeptide” and “protein” are used interchangeably to refer to a polymer of amino acid residues, comprising natural or non-natural amino acid residues, and are not limited to a minimum length. Thus, peptides, oligopeptides, dimers, multimers, and the like are included within the definition. Both full-length proteins and fragments thereof are encompassed by the definition. The terms also include post-translational modifications of the polypeptide, including, for example, glycosylation, sialylation, acetylation, and phosphorylation. Furthermore, a “polypeptide” herein also refers to a modified protein such as single or multiple amino acid residue deletions, additions, and substitutions to the native sequence, as long as the protein maintains a desired activity. For example, a serine residue may be substituted to eliminate a single reactive cysteine or to remove disulfide bonding or a conservative amino acid substitution may be made to eliminate a cleavage site. These modifications may be deliberate, as through site-directed mutagenesis, or may be accidental, such as through mutations of hosts, which produce the proteins or errors due to polymerase chain reaction (PCR) amplification.

As used herein, a “conservative” amino acid substitution refers to the substitution of an amino acid in a peptide or polypeptide with another amino acid having similar chemical properties, such as size or charge. A conservative substitution, as known to one of ordinary skills in the art, refers to a complete replacement of an amino acid residue with a different residue having similar biochemical characteristics, such as size, charge, polarity, etc. For instance, the aromatic Tyrosine may be conservatively substituted with aromatic phenylalanine, or basic Arginine may be conservatively substituted with basic Lysine

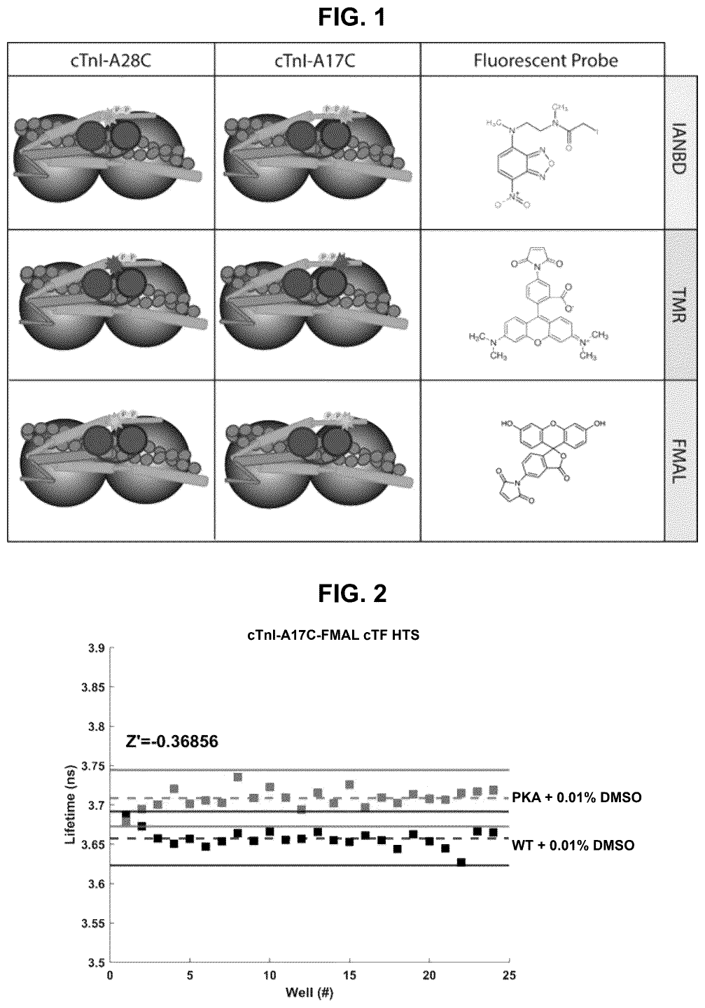

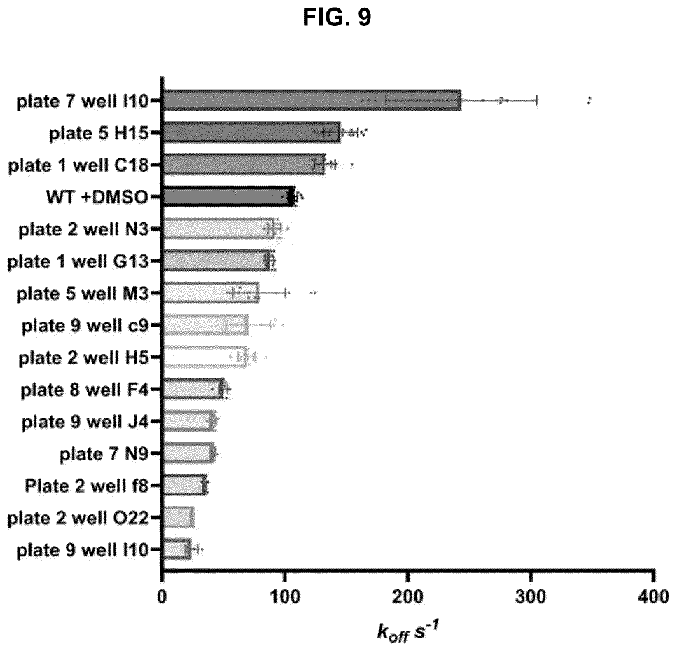

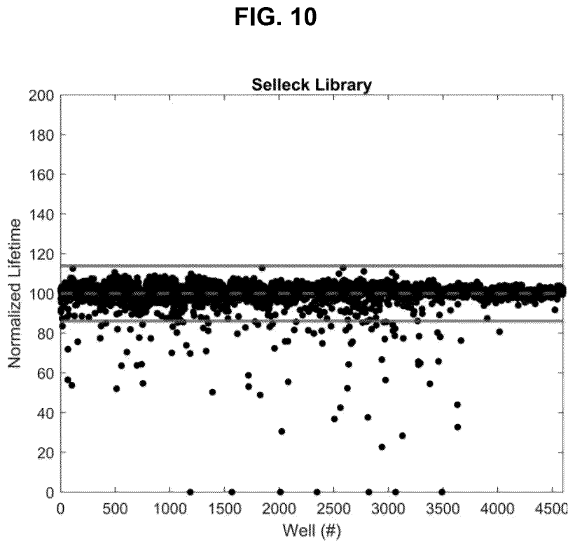

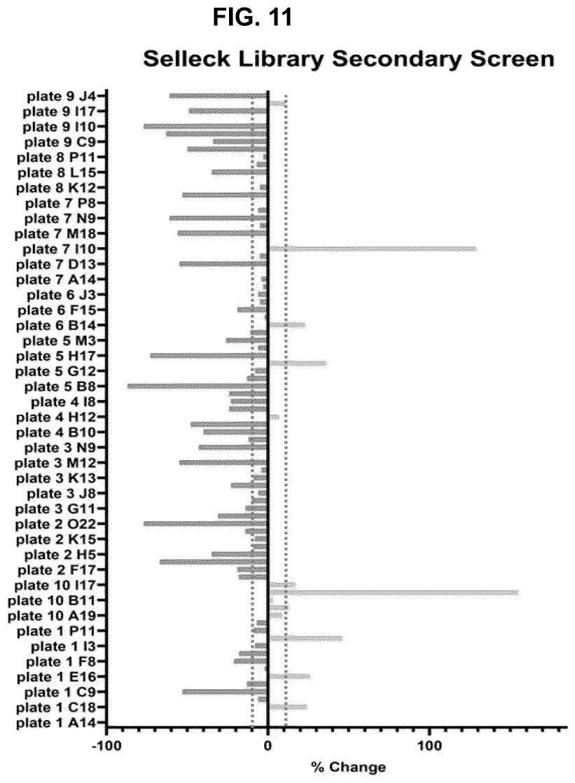

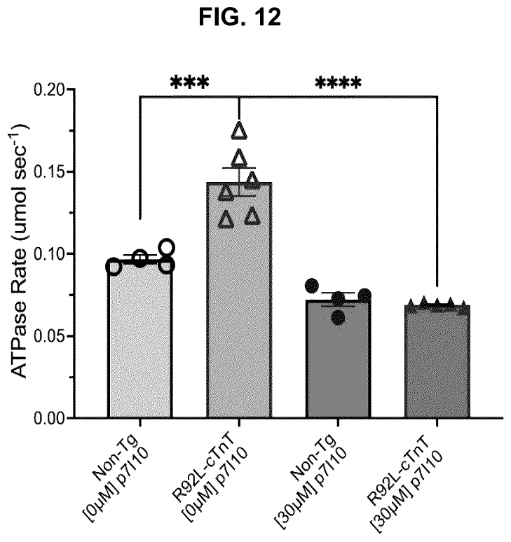

Referring now to FIGS. 1-9, the present invention features compositions and methods for detecting thin filament modulation.

The present invention may feature a recombinant protein comprising a modified human cardiac troponin protein comprising a modification at A28 and a fluorescent dye conjugate to said modification. Likewise, in some embodiments, the present invention features a recombinant protein comprising a modified human cardiac troponin protein comprising a cysteine substitution at A28 and a fluorescent dye conjugate to the cysteine substitution. In some embodiments, the recombinant protein is sensitive to phosphorylation at S2324.

In certain embodiments, the human cardiac troponin protein may further comprise a modification at cysteine 80 (C 80), cysteine 97 (C97), or a combination thereof. For example, the cysteine may be substituted for a serine, an isoleucine, or the like.

Non-limiting examples of fluorescent dyes that may be used in accordance with the present invention include Tetramethylrhodamine-6-maleimide (TMR), fluorescein-5-maleimide (FMAL), N,N′-dimethyl-N-(iodoacetyl)-N′-(7-nitrobenz-2-oxa-1,3-diazol-4-yl)ethylenediamine (IANBD), or the like.

In some embodiments, the present invention features a recombinant protein comprising a modified human cardiac troponin protein comprising a modification at A28 and an N,N′-dimethyl-N-(iodoacetyl)-N′-(7-nitrobenz-2-oxa-1,3-diazol-4-yl) ethylenediamine (IANBD) probe conjugate to said modification. Likewise, in some embodiments, the present invention features a recombinant protein comprising a modified human cardiac troponin protein comprising a cysteine substitution at A28 and an N,N′-dimethyl-N-(iodoacetyl)-N′-(7-nitrobenz-2-oxa-1,3-diazol-4-yl) ethylenediamine (IANBD) probe conjugate to the cysteine substitution. In some embodiments, the recombinant protein is sensitive to phosphorylation at S2324.

The present invention may further feature a reconstituted complete thin filament system comprising a recombinant protein, as described herein. For example, the recombinant protein may comprise a modified human cardiac troponin protein comprising a modification at A28 (e.g., a cysteine substitution) and a fluorescent dye conjugate to said modification. In certain embodiments, the systems herein may further comprise an actin protein, a tropomyosin protein, or a combination thereof. In other embodiments, the present invention may feature a reconstituted complete thin filament system comprising a recombinant protein as described herein, an actin protein, and a tropomyosin protein. In some embodiments, the complete thin filament system responds to biological cues.

In some embodiments, the physiological interactions within the reconstituted complete thin filament system are intact.

### EXAMPLE 1

### Purification and Mutagenesis

The following is a non-limiting example of the present invention. It is to be understood that said example is not intended to limit the present invention in any way. Equivalents or substitutes are within the scope of the present invention.

Probe Descriptions: Engineered cardiac troponin I fluorescent probe at site A28C, using the environmentally sensitive dye IANBD. This probe is reconstituted into the cardiac troponin complex, and then the troponin complex is reconstituted into the cardiac thin filament.

Protein expression and purification: cDNA sequences encoding human cTnT (hcTnT), human cardiac troponin I (hcTnI), human cardiac troponin C (hcTnC), and Ala-Ser α-Tm were inserted into pET3D vectors and provided by J. D. Potter (University of Miami). Single cysteine substitutions were introduced to hcTnl at site A28C via the QuickChange II XL site-directed mutagenesis kit (Agilent Technologies). The TnC (84C) construct was made via substitution of the endogenous cysteine at site 35 with a serine. Each clone was sequenced by the University of Arizona Genetics Core through direct DNA sequencing and verified using the SnapGene Viewer (GSL Biotech LLC). The cTnI, cTnC, and Ala-Ser-tropomyosin plasmids were transformed into BL21 competent cells. While the cTnT plasmid was transformed into Rosetta (DE3) competent cells-Novagen (EMD, Millipore). All transformed cells were streaked onto Luria Broth—ampicillin agar plates and incubated at 37° C. overnight. A single colony from each plate was inoculated into 5-7 mL of LB media and incubated at 37° C. while shaking at 250 rpm for 7 hours. 1 mL of the starter culture was then inoculated into either ZYP medium (1% tryptone, 0.5% yeast, 0.5% (w/v) glycerol, 0.05% glucose, and 0.2% lactose) with 5% 20× P-buffer (1 M Na₂HPO₄, 1 M KH₂PO₄, and 0.5 M (NH₄)₂SO₄), 1 mM MgSO₄, and ampicillin (cTnT, cTnI, and Tm) or overnight Express TB medium (cTnC). The large cultures were grown overnight at 37° C. shaking at 250 rpm. The large cultures were then collected and centrifuged at 4000 rpm for 20 min at 4° C.

cTnI purification: The cTnI bacterial pellets were resuspended in 50 mL Sp-Sepharose buffer (6M Urea, 50 mM Tris, 2 mM EDTA, and 1 mM DTT, pH 7.0). The pellets were then frozen at −80° C. and thawed for sonication. The suspended pellet was sonicated for 30 second bursts with 2 minute pauses for 6 cycles. The resuspended pellet was then centrifuged for 45 minutes at 17,000 RPM to pellet bacterial debris. The supernatant was then kept and loaded into an Sp-Sepharose column (Sigma; packed in a Bio-Rad Econo-Column with a 100 mL bed volume) at a rate of 1.3 mL/min. The column was then washed with 5-7× column volume of Sp-Sepharose buffer and eluted via a linear gradient from 0-0.6M KCl in Sp-Sepharose buffer. Fractions containing cTnI were determined through Coomassie staining of sodium dodecyl sulfate polyacrylamide gel electrophoresis (SDS-PAGE) gel, which were then pooled and dialyzed against 2L TnC affinity buffer (50 mM Tris, 2 mM CaCl2, 0.5 M KCL 1 mM DTT, pH 7.5) for two subsequent dialysis changes for at least 8 hours. Dialyzed protein was then loaded into the TnC affinity column prepared as per the manufacturer's protocol for conjugating proteins to a cyanogen bromide-activated Sepharose 4B gel (Sigma) and packed in an Econo-Column (Bio-Rad). The column was washed with 5-7× column volumes of TnC affinity buffer, and the protein was eluted using both urea and EDTA gradients (0-6 M and 0-3 mM, respectively) in TnC affinity buffer.

WT cTnT purification: cTnT was purified on an Sp-Sepharose column and eluted with a linear gradient of 0-0.6 M KCl as described for cTnI. The fractions containing cTnT were determined through Coomassie staining of SDS-PAGE gel. The fractions with cTnT protein was the dialyzed against 2L of Q-Sepharose buffer (6M Urea, 20 mM Tris, 1 mM EDTA, 1 mM DTT, pH 7.8) for two subsequent dialysis changes for at least 8 hours. The dialyzed protein was recovered and loaded on a Q-Sepharose column (Sigma; packed in a Bio-Rad Econo-Column with a 100 mL bed volume). The column was then washed with 5-7× the column volume of Q-Sepharose buffer and eluted with a linear gradient of 0-0.6M KCl in Q-Sepharose buffer.

WT cTnC purification: cTnC was initially purified on Q-Sepharose columns as described with cTnT. The fractions containing cTnT were determined through Coomassie staining of SDS-PAGE gel. The fractions with cTnT protein was dialyzed against 4L of Phenyl-Sepharose A buffer (50 mM Tris, 1 mM CaCl2, 1 mM MgCl2, 50 mM NaCl, 1 mM DTT, pH 7.5) for four subsequent dialysis changes for at least 8 hours at 4C. During the dialysis process, the room temperature phenyl Sepharose column was regenerated with 5× the volume of de-gassed 30% isopropanol (in ddH₂O) followed by 500 mL of ddH2O. The phenyl Sepharose column was then pre-equilibrated at room temperature, degassed Phenyl Sepharose A buffer with the addition of 0.5M ammonium sulfate. The protein was recovered from dialysis and allowed to warm to room temperature. Once the protein was at room temperature, solid ammonium sulfate was added to a concentration of 0.5M. The protein was then loaded into the Phenyl-Sepharose column (Sigma; packed in a Bio-Rad Econo-Column with a 100 mL bed volume) at a rate of 1.3 mL/min. The column was then washed with 5× the column volume of the Phenyl-Sepharose A buffer with 0.5M ammonium sulfate. The cTnC was then eluted with 500 mL of Phenyl Sepharose C buffer (50 mM Tris, 1 mM EDTA, 1 mM DTT, pH 7.5) at a rate of 1.3 mL/min.

WT Tropomyosin purification and Actin: Ala-ser-Tm bacterial pellets were resuspended in 40 mL of ddH₂O and transferred to a plastic beaker. 4 mg of lysozyme was added to the resuspended pellet and stirred on ice every 5-10 minutes for an hour. The pellet was then frozen at −80° C. and then thawed. Solid NaCl was added to the thawed pellet to a final concentration of 1M and then sonicated on ice for 3 minutes with 3 minutes of rest for 3 cycles. The sonicated pellet was then centrifuged at 17000 RPM for 45 minutes at 4° C. to remove bacterial debris. The supernatant was then collected in a 50 mL conical and boiled for 45 minutes. The sample was then centrifuged for 10 minutes at 17000 RPM at 4° C. The supernatant was collected, and 1M HCL was added dropwise to a pH of 4.4-4.6 to precipitate the tropomyosin. The sample was then centrifuged for 10 minutes at 17000 RPM at 4° C. The supernatant was decanted, and the pellet was resuspended in 1M KCl. KOH was added dropwise to a pH of 7-8 to resuspend the pellet. This process was repeated 3 times in order to obtain purified tropomyosin. F-Actin was isolated and purified from rabbit skeletal muscle as previously described. For all proteins purity was determined through Coomassie staining of the SDS-PAGE gels.
1) Mutagenize the human cardiac troponin I plasmid with the following amino acid substitutions: Alanine 28→cysteine (A28C), Cysteine 80→serine (C80S), Cysteine 97→isoleucine (C97).
2) Transform cTnI-C80S/C97I/A28C, WT cTnC, WT TnT into BL21 competent cells to bacterially express the individual troponin subunit proteins.
3) Purify the individual troponin subunits using the bacterial expression and purification protocol for each corresponding troponin subunit.
4) Take the cTnI-C80S/C97I/A28C protein and dialyze it into a 6M Urea solution overnight
5) Recover the cTnI-C80S/C97I/A28C and incubate it with a 4-molar excess concentration of (tris(2-carboxyethyl)phosphine) (TCEP) at room temperature for 30 minutes to reduce the protein.
6) Take the reduced cTnI-C80S/C97I/A28C and incubate it with an 8-molar excess of the fluorescent probe N,N′-dimethyl-N-(iodoacetyl)-N′-(7-nitrobenz-2-oxa-1,3-diazol-4-yl)ethylenediamine (IANBD) for 4 hours at room temperature.
7) Stop the reaction by adding 5 mM DTT to the sample.
8) Dialyze out the excess unreacted probe with at least 3 2L dialysis changes of at least 8 hours.
9) Recover the now labeled cTnI-C80S/C97I/A28C-IANBD and measure the labeling efficiency. (Only continue with protein >90% labeling efficiency)
10) Take labeled cTnI-C80S/C97I/A28C-IANBD, WT cTnC, and WT cTnI and dialyze into a 6M urea solution.
11) Recombine the troponin subunits at a molar ratio of 1.2:1:1.2 (WT cTnC:cTnI-C80S/C97I/A28C-IANBD:WT cTnT) and dialyze this reconstituted sample against a 6M urea solutions.
12) Progressively lower the Urea concentration from 6M to 4M to 2M to 0M via dialysis changes every 8 hours. This will generate labeled troponin.
13) Dialyze the labeled troponin into the FRET working buffer.
14) Recover the troponin and clarify the sample by spinning on a tabletop centrifuge at 21000 rpm for 10 minutes. After the spin, keep the supernatant.
15) Purify G-actin from rabbit skeletal muscle and bacterially expressed WT alaser-alpha Tropomyosin.
16) Polymerize G-actin to F-actin by adding 0.05M KCl and 2 mM MgCl2 and gently mixing at room temperature for an hour.
17) Dialyze F-actin and WT alaser-alpha tropomyosin into a FRET working buffer overnight.
18) Recover all protein and reconstitute the cardiac thin filament at a ratio of 8.75 uM F-actin, 1.25 uM WT alaser-alpha tropomyosin, and 1 uM labeled troponin by incubating the F-actin and tropomyosin on ice for 40 minutes and then adding in the labeled troponin to the sample and incubating on ice for another 20 minutes.
19) Dilute the sample to the working concentration of luM troponin with TR-FRET buffer and add in 2 mM CaCl2.

### EXAMPLE 2

The following is a non-limiting example of the present invention. It is to be understood that said example is not intended to limit the present invention in any way. Equivalents or substitutes are within the scope of the present invention.

Six different time-resolved fluorescence (TR-F) based assays were tested in order to identify the most sensitive thin filament probe sensitive to the phosphorylation of cardiac troponin I. Site-directed mutagenesis was performed and generated two different protein constructs, either cTnI-A17C or cTnI-A28C. Both protein constructs were labeled with the environmentally sensitive probes N, N′-dimethyl-N-(iodoacetyl)-N′-(7-nitrobenz-2-oxa-1,3-diazol-4-yl)ethylenediamine (IANBD), Tetramethylrhodamine-6-maleimide (TMR), or fluorescein-5-maleimide (FMAL). These labeled troponin I constructs were reconstituted with cardiac troponin C and cardiac troponin T to form the troponin complex. The troponin complexes were then incubated with F-actin and ALA-SER alpha tropomyosin to form the cardiac thin filament. This yielded six independent thin filament systems for testing with the goal of identifying screening assays sensitive to phosphorylation of cardiac troponin I (FIG. 1).

In order to identify which thin filament system is the most sensitive to TnI-Ser23/24 phosphorylation, the cTF systems were phosphorylated with the catalytic subunit of PKA. Fluorescence lifetime measurements were then obtained using a fluorescence lifetime plate reader (fluorescence innovations) of the WT and phosphorylated systems. Once fluorescence lifetimes were obtained, Z′ scores (eq. 1) were computed to assess the fidelity of the assay for high throughput drug screening.

$${Z}^{′} = 1 - ( 3 \frac{\vert {σ}_{A} - {σ}_{B} \vert}{\vert {μ}_{A} - {μ}_{B} \vert} ) \qquad Eq . 1$$

An assay with a Z′ value greater than 0.5 is deemed useful for drug screening. The assay with the largest Z′ value was continued for testing with the 3057 compound Selleck FDA-approved drug library. Stopped flow calcium dissociation kinetics were then employed for the secondary screening assay to validate the fidelity of the primary screen in identifying small molecules that modulate the rate of calcium dissociation from the thin filament.

None of the A17C-based cardiac troponin C (cTF) constructs displayed sensitivity to cardiac troponin I (cTnI) phosphorylation, as evidenced by Z′ values consistently less than 0.5 (refer to Table 1). However, both cTnI-A28C-FMAL and cTnI-A28C-IANBD exhibited sensitivity to TnI phosphorylation, with only cTnI-A28C-IANBD deemed suitable for high-throughput drug screening purposes. Notably, cTnI-A28C-IANBD cTFs demonstrated the highest sensitivity to Protein Kinase A (PKA) with a Z′ value of 0.86, indicating its excellence for high-throughput screening (see Table 1). Initial screening utilizing the Selleck FDA-approved drug library yielded a 0.85% hit rate. Further, among the hits proceeding to secondary screening, 75% of the small molecules were found to alter calcium dissociation kinetics. In conclusion, the cTnI-A28C-IANBD cTF system proved successful in identifying small molecules capable of modulating calcium dissociation rates within the thin filaments, suggesting its utility in drug discovery efforts targeting cardiac function modulation.

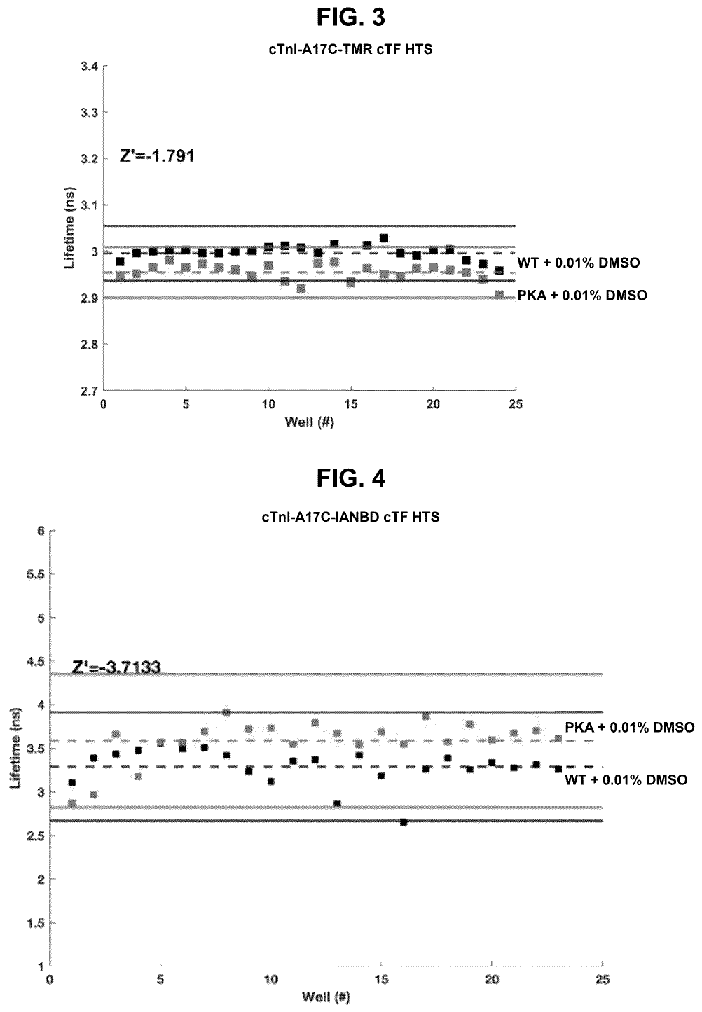

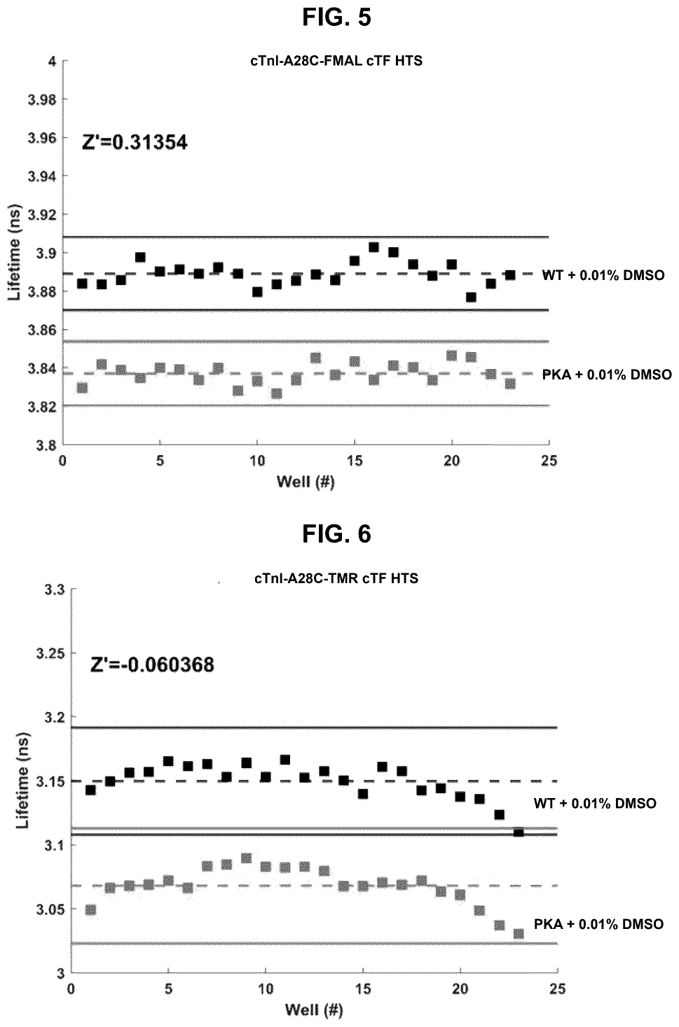

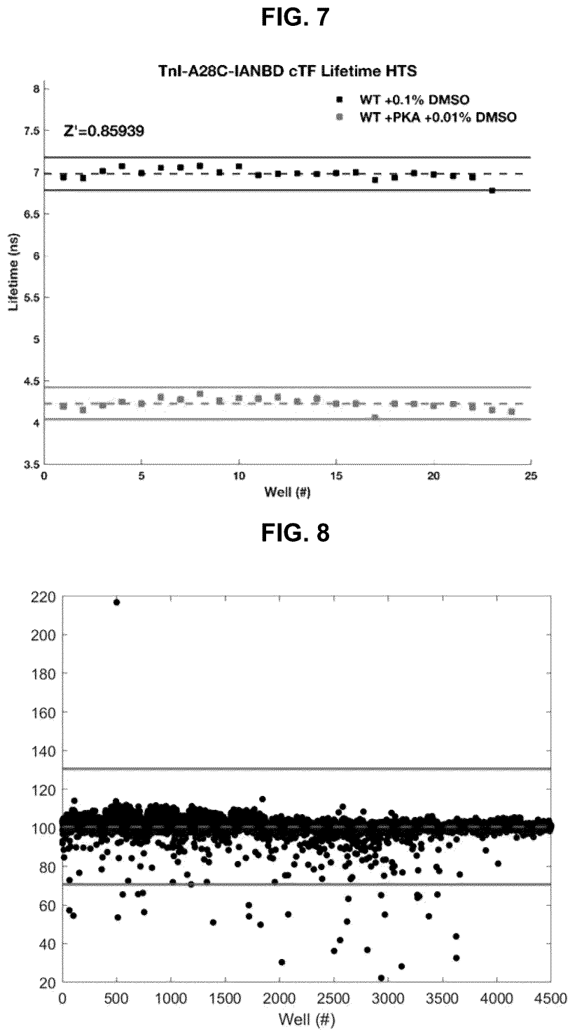

TABLE 1

| cTF system | Z′ value | Corresponding FIG. |
|---|---|---|
| cTnI-A17C-FMAL | −0.37 | FIG. 2 |
| cTnI-A17C-TMR | −1.79 | FIG. 3 |
| cTnI-A17C-IANBD | −3.71 | FIG. 4 |
| cTnI-A28C-FMAL | 0.31 | FIG. 5 |
| cTnI-A28C-TMR | −0.06 | FIG. 6 |
| cTnI-A28C-IANBD | 0.86 | FIG. 7 |

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A recombinant protein comprising:
a. a modified human cardiac troponin protein comprising a modification at A28; and
b. a fluorescent dye conjugate to the modification at A28;
wherein the recombinant protein is sensitive to phosphorylation at S2324.

2. The recombinant protein of claim 1, wherein the modification at A28 is a cysteine substitution.

3. The recombinant protein of claim 2, further comprising a fluorescent dye conjugate to the cysteine substitution.

4. The recombinant protein of claim 1, wherein the human cardiac troponin protein further comprises a modification at cysteine 80 (C 80), cysteine 97 (C97), or a combination thereof.

5. The recombinant protein of claim 4, wherein the cysteine is substituted for a serine or an isoleucine.

6. The recombinant protein of claim 1, wherein the fluorescent dye is N,N′-dimethyl-N-(iodoacetyl)-N′-(7-nitrobenz-2-oxa-1,3-diazol-4-yl)ethylenediamine (IANBD), Tetramethylrhodamine-6-maleimide (TMR), or fluorescein-5-maleimide (FMAL).

7. A recombinant protein comprising:
a. a modified human cardiac troponin protein comprising a cysteine substitution at A28; and
b. an N,N′-dimethyl-N-(iodoacetyl)-N′-(7-nitrobenz-2-oxa-1,3-diazol-4-yl) ethylenediamine (IANBD) probe conjugate to the cysteine substitution,
wherein the recombinant protein is sensitive to phosphorylation at S2324.

8. A reconstituted complete thin filament system comprising a recombinant protein, the recombinant protein comprising:
a. a modified human cardiac troponin protein comprising a modification at A28; and
b. a fluorescent dye conjugate to the modification at A28;
wherein the recombinant protein is sensitive to phosphorylation at S2324.

9. The system of claim 8, wherein physiological interactions within the reconstituted complete thin filament system are intact.

10. The system of claim 8, further comprising an actin protein, a tropomyosin protein, or a combination thereof.

11. The system of claim 10, wherein the system responds to biological cues.
